(19) United States
(12) Patent Application Publication
(10) Pub. No.: US 2023/0120321 A1
(43) Pub. Date: Apr. 20, 2023

(54) THREE-DIMENSIONAL INTRAOCULAR LENS SCAFFOLD AND ADD-IN LENS COMBINATION AND METHODS OF IMPLANTATION

(71) Applicant: ANEW IOL TECHNOLOGIES, INC., LAKE GENEVA, WI (US)

(72) Inventors: GREGORY LEE FLETCHER, NAPLES, FL (US); ANNA S. HAYES, BLUE HILL, ME (US); JEFFREY SCOTT CALLAHAN, BLUFF FITY, TN (US); JOSEPH DONALD CALLAHAN, BLUFF FITY, TN (US)

(73) Assignee: ANEW IOL TECHNOLOGIES, INC., LAKE GENEVA, WI (US)

(21) Appl. No.: 17/968,073

(22) Filed: Oct. 18, 2022

Related U.S. Application Data
(60) Provisional application No. 63/256,755, filed on Oct. 18, 2021.

(51) Int. Cl. A61F 2/16

(52) U.S. Cl. CPC A61F 2/1601; A61F 2/1616; A61F 2/1621; A61F 2/1637; A61F 2/1645; A61F 2/1648; A61F 2002/1683

(57) ABSTRACT

Devices and methods for replacing a human lens after cataract surgery. The device is an insert for the eye capsule and is formed of two or more rings that are connected to one another. A primary lens is affixed to the insert. A secondary add-in lens can be added to the insert in a subsequent surgery to correct, or further optimize, the optical results obtained with the initial surgery.

### RELATED APPLICATIONS

This application claims priority to U.S. provisional patent application Ser. No. 63/256,755 filed on 18 Oct. 2021.

### BACKGROUND OF THE INVENTION

### 1. Field of the Invention

The invention provides structural mechanisms intended to be placed within the natural lens capsule of the eye following surgical removal of the natural crystalline lens.

### 2. Description of the Background

A cataract is not a disease of the eye, but a natural condition of the eye normally related to aging. The eye produces lens epithelial cells on the anterior capsule of the lens. These cells migrate to the fornix, or equator, of the lens where they convert into lens cortical material. As the body ages, the lens, contained within the capsule which is of limited volume, is impacted by the cortical material and grows denser. Eventually the lens density is such that the lens material becomes increasingly opaque (cataractous) and can eventually completely block light transfer to the retina, causing blindness. Before then vision becomes compromised and patients often elect cataract surgery.

Cataract surgery first entails removal of the natural, crystalline lens (now cataractous) from the capsule. Before advancements leading to current medical techniques, the lens capsule was left empty (aphakic), and the cataract patient had to wear very thick glasses to be able to see. Cataract surgery involving lens replacement inside the vacated capsule began in the early 1950s in England with the first implantation of a PMMA lens. Since then the surgical process for cataract removal and replacement has advanced significantly using more advanced lens materials while utilizing predominately flat, two-dimensional (2-D), intraocular lenses (IOLs) with the caveat that some postsurgical consequences of cataract procedures continue to degrade patients' visual acuity. Even as new techniques make cataract surgery safer, faster, and more consistent in terms of results, these 2-D IOLs do not yet adequately address capsular fibrosis and its consequences, nor do they fully mitigate posterior capsule opacification (PCO) and its attendant consequences and risks, nor do they always result in desired refractive results, i.e., clarity of vision over a target range of distances, and/or satisfying many patients' expectations that they will be without spectacles following surgery. Most cataract IOLs that are currently commercially available (2-D designs), especially the lenses that are sold as “premium” lenses because they purport to offer better vision, do not deliver, or tend to lose over time, their promised attributes. Resultant vision impairment can have a significant impact on a person's quality of life.

Further, patients that have astigmatism may have continuing vision impairment, even after having a prior art IOL implanted.

### SUMMARY OF THE INVENTION

The present invention overcomes the problems and disadvantages associated with current surgical strategies and lens designs by utilizing a three-dimensional (3-D) intraocular lens (IOL) for the initial surgical implantation for mitigating post surgical adverse effects that cause patients to not initially achieve, and/or to subsequently lose, targeted visual acuity. The 3-D IOL can then serve as a scaffold in combination with a secondary optic subsequently implanted for correction or optimization of results from the initial surgery.

The scaffold of the present invention comprises two or more rings connected by a two or more pillars. The rings are formed in such a manner that the secondary lens can be situated within the scaffold. The scaffold may also have fenestrations cut into the pillars to provide for circulation of the aqueous throughout the volume within the scaffold and for affixing the add-in lens(es).

The secondary optic can be positioned within the scaffold at varying positions along the optical axis and can be rotated to a target position.

The present invention further provides methods for improving refraction (focus) and for addressing astigmatism (vision impact of a non-spherical cornea) after an initial cataract surgery. Specifically, for the initial surgery, one structural mechanism comprises two or more rings connected by a series of pillars and struts or platforms and a primary optic that form a cylindrical volume (a scaffold) that provides for the subsequent implantation, if deemed necessary, of a second mechanism, i.e., one or more additional optics that may be inserted and affixed within the scaffold.

The present invention teaches a unique combination of products with an integral set of critically important ophthalmological attributes and methods of execution assembled in a manner heretofore unavailable to ophthalmologists. The products, attributes and methods of the present invention are carefully delineated within this application and provide a powerful combination compared to similar prior art products and methods in the industry.

### BRIEF DESCRIPTION OF THE DRAWINGS

FIG. 1 illustrates the general anatomy of a healthy human eye, especially the lens, optical axis, capsule, zonules and ciliary body.

FIG. 2 illustrates the human eye, affected by a cataract.

FIG. 3 illustrates the anatomy of the adult human lens.

FIG. 4 further depicts the adult human lens, providing terminology pertinent to cataract development and the development of anterior capsular opacification and posterior capsular opacification.

FIGS. 5A-5C demonstrate prior art two-dimensional intraocular lenses that are most commonly used in cataract treatment.

FIG. 6A is a perspective view of a prior art 3-D intraocular lens with horizontal fenestrations between the posterior ring and the optic.

FIG. 6B is an overhead view of the lens of FIG. 6A.

FIG. 6C is a cross-sectional view of the lens of FIG. 6B taken along the line 6C-6C.

FIG. 7 is a perspective views of a scaffold according to the present invention demonstrating vertical fenestrations through the anterior and posterior rings and horizontal fenestrations.

FIG. 8 is an overhead view of the scaffold shown in FIG. 7.

FIG. 9 is a planar view of the scaffold of FIGS. 7-8.

FIG. 10 is a cross-sectional view of the scaffold of FIG. 8 taken along the line 10-10 of FIG. 8 showing combined horizontal and vertical fenestrations.

FIG. 10A is a close-up sectional view of the scaffold shown in FIG. 10.

FIG. 10B is a cross-sectional view of the scaffold of FIG. 8 along the line 10B-10B depicting only a horizontal fenestration.

FIG. 11 is an alternate embodiment of a scaffold according to the present invention.

FIG. 12 is a planar view of the scaffold of FIG. 11.

FIG. 13 is an overhead view of the scaffold shown in FIG. 11.

FIG. 14 is a cross-sectional view of the scaffold of FIG. 14 taken along the line 14-14 of FIG. 13.

FIG. 15 demonstrates the second embodiment of an add-in lens according to the present invention, having a gear toothed design.

FIG. 16 is an overhead view of an add-in lens of the present invention that can be used in connection with the scaffold of the present invention.

FIG. 17A is a cross-sectional view of a convex add-in lens of the present invention.

FIG. 17B is a cross-sectional view of a concave add-in lens of the present invention.

FIG. 17C is a cross-sectional view of a flat add-in lens of the present invention.

FIGS. 18 and 19 show alternate add-in lenses according to the present invention having differing arrangements of anchoring mechanisms used to secure the lenses within a scaffold.

FIG. 20 is an overhead view of the scaffold of FIG. 7 in combination with the lens of FIG. 16.

FIG. 21A is a cross-sectional view of a convex add-in lens as shown in FIG. 17A in combination with the scaffold of the present invention.

FIG. 21B is a cross-sectional view of a concave add-in lens as shown in FIG. 17B in combination with the scaffold of the present invention.

FIG. 21C is a cross-sectional view of a flat add-in lens as shown in FIG. 17C in combination with the scaffold of the present invention.

FIGS. 22-23 demonstrate steps during implantation of a device according to the present invention.

FIGS. 24-28 demonstrate cross-sectional views of present invention in various forms within the eye capsule.

FIG. 24 demonstrates a planar view of a scaffold of the present invention inserted within the anterior eye segment.

FIG. 25 demonstrates a cross-sectional view of the scaffold of FIG. 24 before insertion of an add-in lens.

FIG. 26 further demonstrates a cross-sectional view of a flat add-in secondary lens inserted into the scaffold of FIG. 24, as also depicted in FIG. 21C

FIG. 27 is a cross-sectional view of the scaffold of FIG. 25 with the addition of a convex add-in secondary lens, as is also depicted in FIG. 21A.

FIG. 28 demonstrates an exterior view of an alternate embodiment of the present invention within the eye capsule.

FIGS. 29A and 29B demonstrate an anterior view of an add-in lens of the present invention having rotational capability.

FIG. 30 shows a 2-D prior art device after being inserted into the eye.

FIG. 30A depicts the collapsing of the eye capsule onto the 2-D prior art device of FIG. 30.

FIG. 31 shows a device of the present invention with a flat add-in secondary lens after being inserted into the eye.

FIG. 32 provides comparative slit-lamp, anterior view, photographic results of a rabbit study after six months, comparing a prior art device (32B) to a scaffold of the present invention without an add-in secondary lens (32A).

FIG. 33 provides comparative results in post-mortem dissection posterior views of a rabbit study after six months, comparing a prior art device (33C, 33D) to a scaffold of the present invention without an add-in secondary lens (33A, 33B).

FIG. 34 provides results of a scaffold of the present invention without an add-in secondary lens of a single patient from a human clinical trial study after 36 months, showing two anterior views.

FIG. 35A portrays the add-in lens in a slit-lamp anterior view from the results of a five-week rabbit study following the implantation of a scaffold with a primary lens of the present invention followed by the implantation of an add-in lens two-weeks later.

FIG. 35B shows the scaffold of FIG. 35A in a post-mortem dissection posterior view.

FIG. 35C depicts a lateral view MRI of the primary lens of the scaffold at the bottom and an add-in convex lens at the top.

FIG. 36 depicts graphs of optical performance from a human clinical study 36 months following implantation of an implant according to the present invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.
I. Introduction
II. Overview
III. Scaffold
IV. Add-In Optic Lenses
V. Scaffold and Add-In Lens in Combination
VI. Methods of Implantation
VII. Benefits
VIII. Results

I. Introduction

The present invention provides improvements in treating cataracts and the symptoms associated with cataracts, by delivering superior optical solutions that are generally provided via a scaffold with a lens and/or add-in lenses. The advantages will be discussed below.

II. Overview

It has surprisingly been discovered that the scaffold of the present invention mitigates post-surgical adverse effects that cause patients to lose visual acuity. The design teaches that maintaining an open capsule, allowing superior circulation of the aqueous humor and minimizing fibrosis compared to prior art inserts can preserve visual acuity and lower the risk of post-operative adverse consequences. Moreover, the scaffold allows for the attachment of an add-in lens, which provides a mechanism for an ophthalmologist to reliably perfect the refractive and astigmatic corrections for each patient, thus making cataract surgery even safer and more effective.

The invention addresses a structural mechanism (the scaffold) intended to be placed within the natural lens capsule of the eye consisting of two or more rings connected by a series of pillars and struts or platforms to which one or more optics may be affixed by means of several attachment (docking) devices. The functional attributes of the scaffold are preferably to keep the capsule open and properly extended; to allow circulation of the aqueous humor throughout the capsule including the entire critical circumferential equatorial portion of the capsule that interconnects with the zonules and, in turn, to the ciliary body; to prevent or minimize unnecessary capsular fibrosis; to provide a 360-degree barrier to harmful epithelial cell migration at the anterior and posterior scaffold ring interfaces with the capsule that, in combination with aqueous circulation, virtually eliminates posterior and anterior capsule opacification; to provide for a healthy interconnection to the accommodative characteristics of the eye; to provide for fit to varying capsule sizes; to provide extended depth of focus via the primary lens; to allow implantation of a primary lens optic; to allow secondary and tertiary implantation(s) of add-in optics or lenses, and to allow explantation of optics for replacement if necessary while greatly diminishing explantation risk. The 3-D scaffold draws from and relates to U.S. Pat. No. 9,439,735 entitled “Haptic Devices for Intraocular Lens,” and U.S. Pat. No. 10,010,405 entitled “Haptic Devices for Intraocular Lens,” both are incorporated in their entirety.

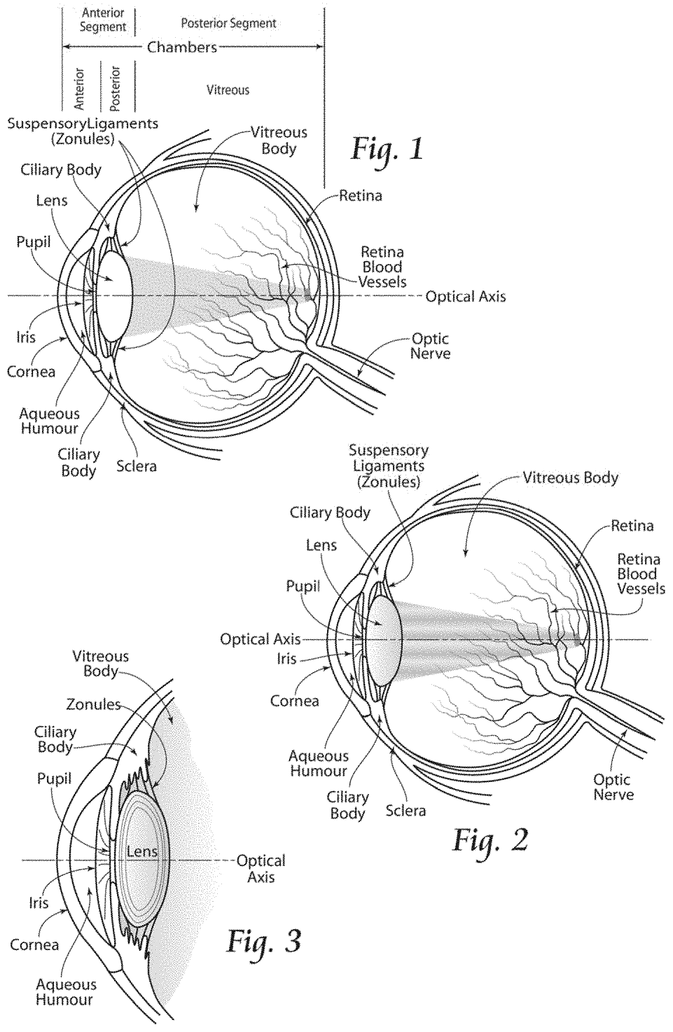

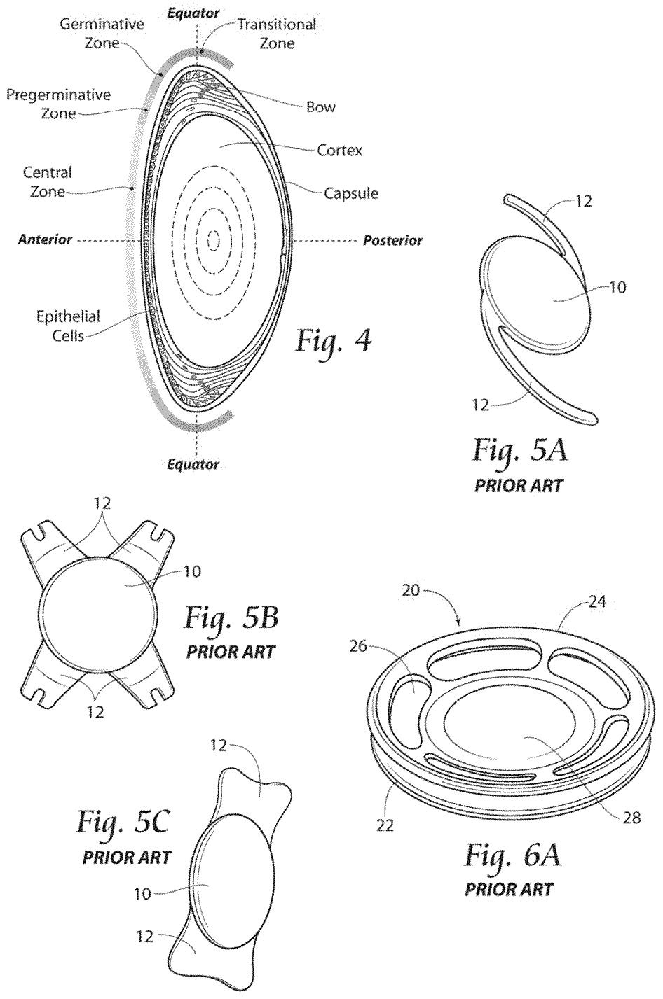

FIGS. 1-4 demonstrate the general anatomy of the eye. As light enters into the eye through the cornea and lens that together refract light rays on the retina for proper focus and vision. The lens is clear, which allows for light to enter the eye. However, as demonstrated in FIGS. 2 and 3, a cataract may form in the eye, which clouds the lens, causing vision issues.

To address these vision issues, the lens is removed and a replacement lens is implanted within the eye. Typical prior art implants are shown in FIGS. 5A-5C, which generally consist of a lens 10 and attachment arms (haptics) 12 that will be affixed within the eye capsule where the lens previously was. These prior art lenses, which have a generally flat arrangement, can be considered a two-dimensional (2-D) intraocular lens (IOL). Because of their lack of depth, following surgery, the capsule of the lens typically will collapse inwardly as the eye heals (See FIG. 30). With the lack of depth of these prior art devices, the capsule will collapse on the 2-D surfaces of these devices, precluding aqueous circulation, with resultant fibrosing of the capsule to all lens components and to the capsule itself. This introduces a host of negative consequences discussed elsewhere in this application.

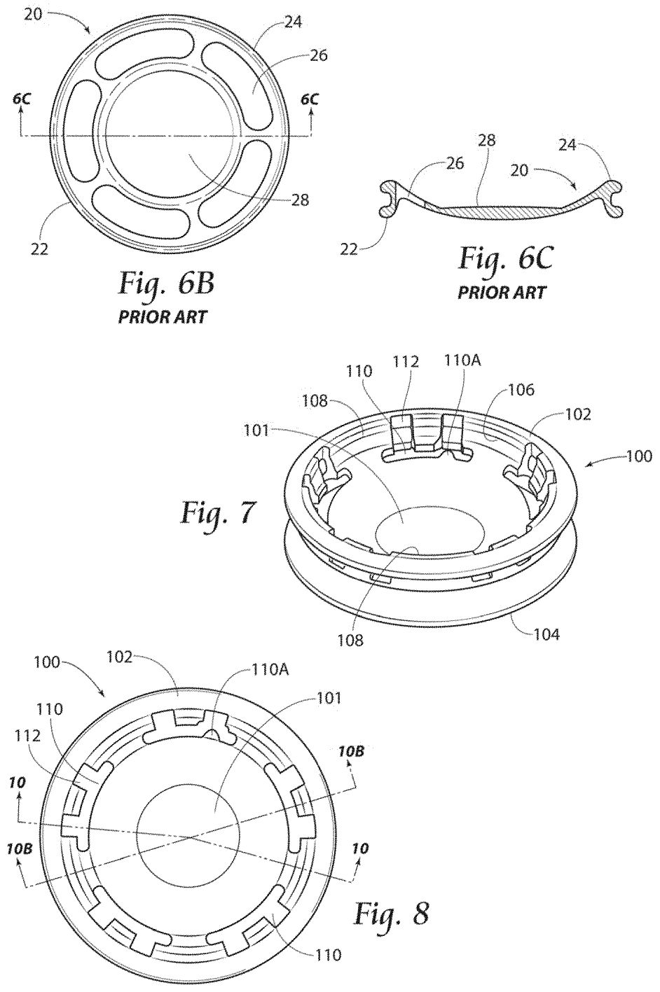

FIG. 6A is a perspective view of another prior art intraocular lens 20. The three-dimensional (3-D) lens 20 comprises a single primary lens 28 and annular rings with an anterior ring portion 24 and a posterior ring portion 22 and horizontal fenestrations 26 at the intersection of the optic and the posterior ring 22. The annular anterior ring portion 24 and annular posterior ring portion 22 provide separation between the anterior and posterior capsular surfaces (See FIG. 6C) and permit aqueous circulation in front of, and behind, the primary lens 28. The lens 20 is an improvement compared to the other discussed prior art implants, but, as discussed below, the present invention provides significant further improvements over these prior art implants.

III. Scaffold

Improving vision through lens replacement not only requires a properly positioned implant, but also requires that the ocular capsule is kept clear after implantation. Particularly, an implant should block or impede particulates that may migrate into the capsule, as well as provide rinsing of the capsule by allowing circulation of aqueous humor throughout the capsule. The structure of the present invention provides improvement of these features over the prior art.

FIGS. 7-10B show an IOL scaffold 100 according to the present invention. The scaffold comprises an anterior ring 102 and a posterior ring 104. The scaffold also has one or more intermediate rings 106. The scaffold 100 also comprises a plurality of pillars 108 that extend from the anterior ring 102 to the posterior ring 104. The pillars 108 are preferably shaped and sized so that the scaffold 100 will fit effectively into the vacated ocular capsule bag. For example, as shown in FIG. 9, the pillars 103 have a curvilinear design. The pillars 108 also have fenestrations 110 and 112 which permit aqueous circulation throughout the capsule subsequent to surgery (via the surgically-created circular hole, or capsulorrhexis or rhexis, in the anterior of the capsule behind the iris through which the crystalline lens is removed and the scaffold is implanted).

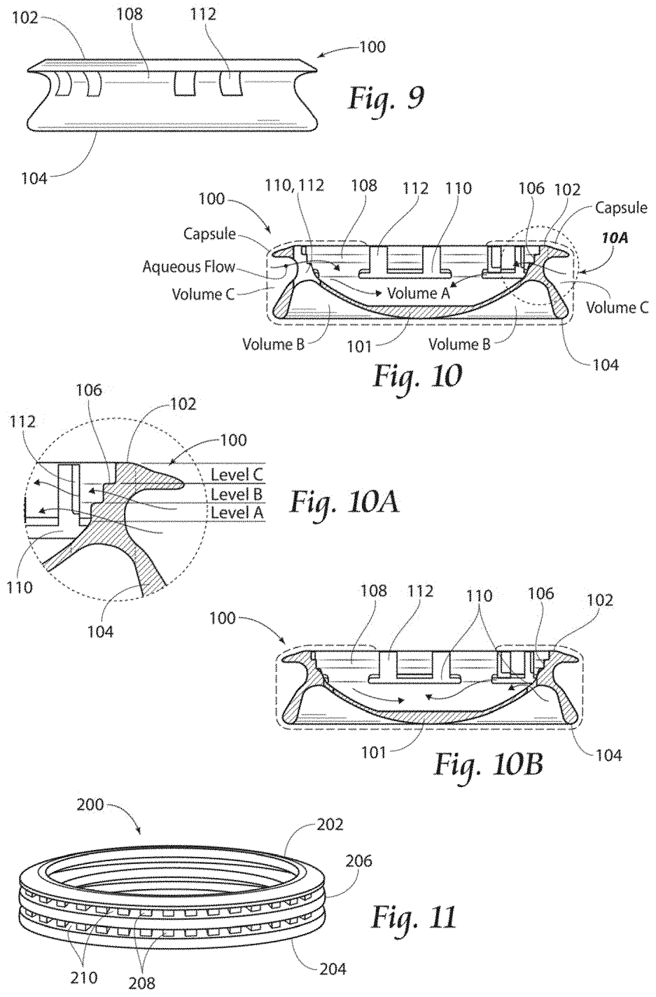

The pillars 108 provide a structure so that the anterior ring 102 and the posterior ring 104 are positioned away from one another, so that the scaffold 100 can be considered a three-dimensional (3-D) structure, which will prevent the ocular capsule from collapsing onto itself and onto the primary lens 101 after surgery, as depicted in FIG. 10. This allows aqueous circulation on either side of the primary lens 101 within the volume formed between the anterior ring 102 and the posterior ring 104 plus the entire equatorial volume C of the lens (see FIG. 10), with aqueous circulation critical in this area. It should be understood that any design of the scaffold having spaced apart rings could be considered a 3-D scaffold according to the present invention.

FIG. 10 is a cross-sectional view of the scaffold 100 and delineates the volume that is formed by the scaffold 100 within the capsule. Volume A would define the portion between the iris and the primary lens, Volume B would be the portion between the primary lens and the posterior capsule, and Volume C would be the volume including the equatorial portion of the capsule. The design of the scaffold 100 allows for each of the volumes A, B, and C to remain clear after implantation by providing a structure for both blocking particulate migration and allowing aqueous humor flow throughout the capsule. Further, the anterior ring 102 has material and design flexibility that, in concert with the pliability of the capsule, allows the scaffold of the present invention to fit within capsules of varying sizes.

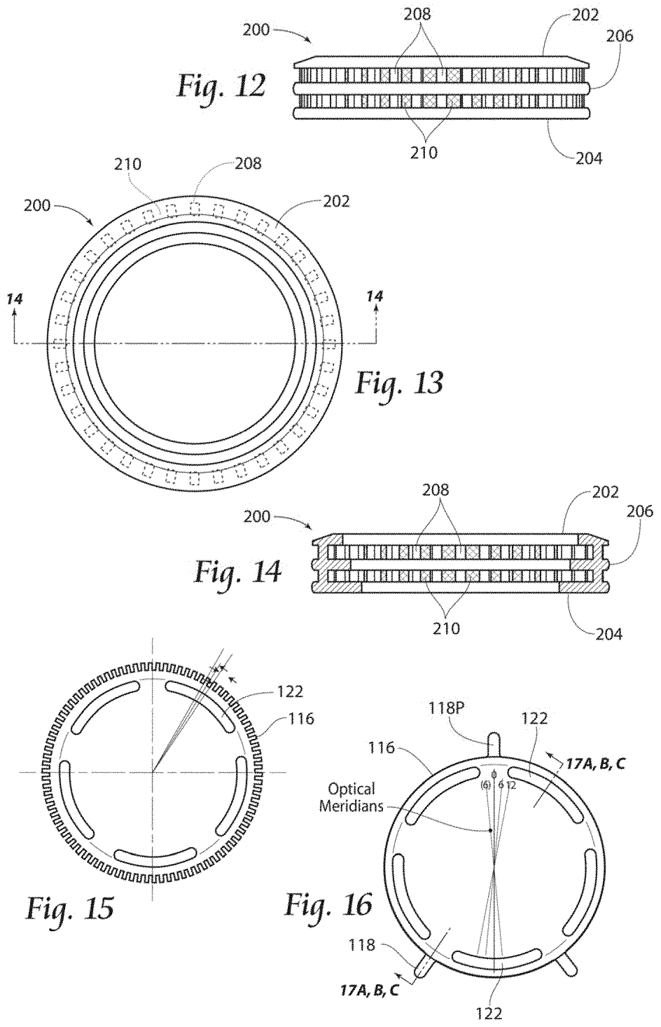

FIGS. 11-14 show another scaffold 200 according to the present invention. The scaffold 200 comprises an anterior ring 202 and a posterior ring 204, as well as an intermediate ring 206 with a diameter less than rings 202 and 204 to permit equatorial aqueous circulation. As with the scaffold 100, the posterior ring 202 and the anterior ring 204 of the scaffold 200 are positioned apart from one another with a plurality of pillars 208, providing a 3-D structure for the scaffold 200. A plurality of fenestrations 210 are located between the pillars 208 that allow aqueous circulation through the scaffold 200 when implanted.

Referring to FIGS. 13 and 14, the three rings 202, 204, and 206, allow for three different levels A, B, and C, similar to those shown with respect to scaffold 100 (See FIG. 10A), that an add-in lens 116 can be positioned, similarly as described for the scaffold 100. The rings are preferably parallel to one another. Likewise, the scaffold 200 allows for the rotation of an add-in lens such as depicted in FIG. 15.

The 3-D IOL scaffold of the present invention is preferably manufactured of any suitable material compatible with the human eye, and in such manner as is consistent with relevant medical device regulations. The scaffold pillars may be of the same material as the scaffold rings or they may be of different materials. The anterior ring may be of the same material as the posterior ring, or it may be of a different material. Likewise, the docking mechanisms may contain different inserted materials than the scaffold pillars, and any portion of the device may be formulated so as to provide slow-release drug delivery of specific pharmaceutical formulations targeting particular diseases of the eye. Compatible materials include, but are not limited to, polymethylmethacrylate, hydrophilic acrylic, hydrophobic acrylic, silicone, or other materials used for Intraocular Lenses (IOLs).

IV. Add-In Optics Lens

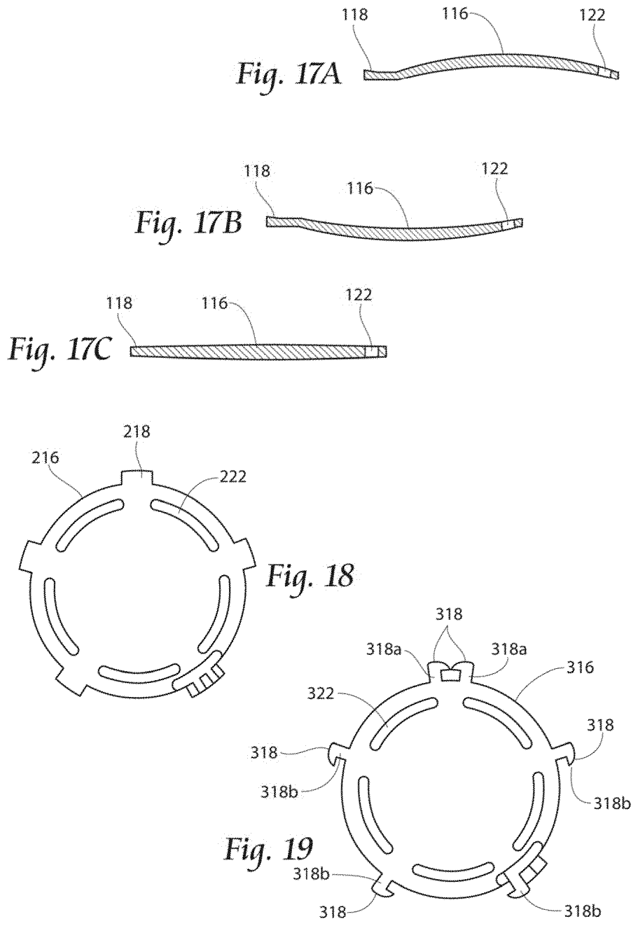

Along with the scaffold, the present invention also incorporates an additional add-in lens 116, as shown in FIG. 16. The lens 116 has haptic arms 118 that extend outwardly from the lens. Three arms 110 are shown. As will be discussed below, the arms 118 provide anchoring mechanisms when inserted into a scaffold 100, 200 of the present invention. The lens also has openings 122 that will permit aqueous circulation when implanted. Depending on the patient's corrective needs, the add-in lens 116 can be of varying curvatures, such as convex (FIG. 17A), concave (FIG. 17B) or flat (FIG. 17C). Alternatively, an add-in lens could be developed as depicted in FIG. 15 that would integrate with tabs in the scaffold 100, 200 thus also providing precise rotational options and optics of various curvatures and optical powers.

It is understood that the add-in lens may be adapted as is necessary for a particular use. As such, FIG. 18 provides an alternate add-in lens 216. The securing devices are in the form of haptic tabs 218, which also assist in securing the lens 216 to a scaffold of the present invention. In a similar fashion, lens 316 of FIG. 19 has a plurality of anchors 318 to secure the lens 316. The anchors 318 preferably comprise a pair of primary anchors 318a that would encircle a pillar of a scaffold of the present invention to lock the lens 316 in place. The lens 316 also has a plurality of secondary anchors 318b with sample angulation to secure behind the scaffold pillars.

It should be understood that the lenses of the present invention could have a varying number of anchoring devices, as demonstrated in FIGS. 15, 16, 18, 19. These securing devices allow the lens to be secured to the scaffold, which is discussed, below.

V. Scaffold and Add-In Lens in Combination

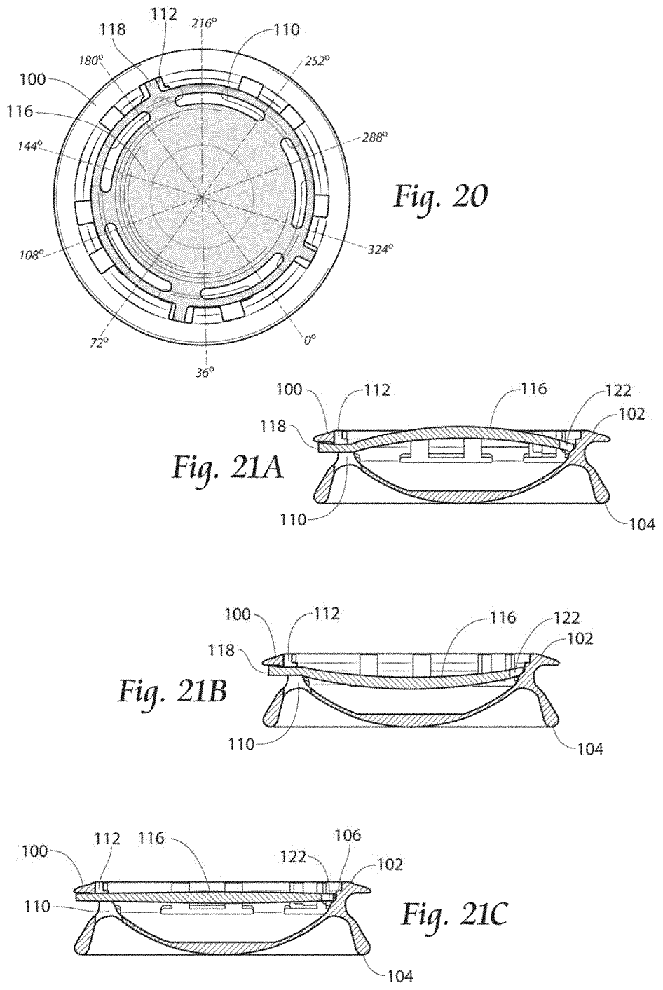

The attributes of the present invention are further enhanced with the combined use of a scaffold and an add-in lens according to the present invention. FIG. 20 demonstrates such an arrangement, showing an overhead view of the lens 116 (FIG. 16) being inserted into the scaffold 100 (FIG. 8). The haptic arms 110 are inserted into the vertical fenestrations 112 of the scaffold so that the lens 116 is anchored to the scaffold 100. As previously noted, the lenses can be of varying curvatures, such as convex (FIG. 21A), concave (FIG. 21B) or flat (FIG. 21C). The arrangement still allows for aqueous circulation when inserted.

The lens 116 will be designed with a diameter so that it will adequately nestle within whichever of the intermediate rings 106 (also exemplified as levels A, B, in FIG. 10A) is determined to address the patient's corrective needs. That is, the proper positioning of the lens 116 on the properly identified level of the intermediate ring 106 will optimize the optical results of the patient. In concert with lens optical design, these distance options allow for considerable optical optimization.

The arrangement depicted in FIG. 20 also allows for rotation of the lens 116 within the scaffold. The lens can be rotated in X-degree increments to permit sufficient placement latitude for toric lenses for accurate treatment of astigmatism at the time of surgery. Optimal rotation increments (represented by X) will be determined by future industry practice as concurrence is still evolving. As an example, combined with the five horizontal fenestrations of the scaffold are ten vertical fenestrations that permit insertion of haptics tabs 118 of add-in 116 (FIG. 16). Because toric lens powers are, in part, described as a meridian across the entire diameter of the lens, and because the 10 vertical fenestrations are asymmetrical, there are 10 meridians that will intersect any 180-degree arc of the optic curvature yielding average rotational options of 18 degrees. The point haptic 118P of add-in 116 (FIG. 16) is generally coincident with the primary meridian. An array of offset meridians (e.g. 6-degrees and 12-degrees from the primary) can provide a total of 30 rotational positions (likely sufficient to address astigmatism correction requirements). Alternatively, tabs could be located on an interior ring of a scaffold, which would be inserted into openings 112 located on the add-in such as those on the gear-tooth lens design depicted as the alternate embodiment in FIG. 15.

As an alternative arrangement, FIGS. 11-14 describe the scaffold 200 comprising the three rings 202, 204, 206 parallel to each other and separated by pillars 208. Fenestrations 210 are indicated by the shaded areas. Optics may be located between the scaffold rings 202, 204, 206, permitting placement of primary and add-in optics with anchor tabs in the fenestrations 230. The scaffold 200 could provide a multitude of increments, so that the lens 116 could be rotated in sufficiently small increments.

The add-in lens of the present invention could have other anchoring arrangements, e.g. FIGS. 18 and 19. In these instances a curvilinear edge of the lens 216 or 316 could potentially be secured and locked to one or more of the pillars 108, with the disclosed tabs 218 or anchors

The anchoring devices of the add-in lens may be planar with the scaffold supports or may be angled to position the lens optic anterior or posterior to the scaffold supports. There may be two or more anchoring devices for the lens, and preferably, at least three haptic arms will provide the most stable positioning yet still provide for easy insertion of the optic into the scaffold.

In all embodiments of the device, several features remain constant. These include the placement of the anterior and posterior rings such that the anterior ring fits against the anterior capsule, and the posterior ring fits against the posterior capsule, while in both cases providing a full 360-degree barrier to undesirable epithelial cell migration. Likewise, spacing of the rings apart from each other by means of a series of scaffold pillars, and the spacing of the pillars to create fenestrations between the pillars allowing circulation of the aqueous humor throughout the capsule—including the critical equatorial area where the zonules interconnect with the capsule is constant in the various designs. Other features may be modified based upon the intended purpose of that particular device, such as: the rings may or may not contain a square edge at one or more locations; there may be a third ring or fourth ring located at some point in the pillar network so as to provide for stable functionality of flexible or accommodating pillar structures, or the pillars could be curved convexly or concavely.

Further, the scaffold of the present invention may comprise two or more rings and any number of pillars to provide for suitable support of one or more optics while allowing ample circulation of the aqueous humor throughout the capsule. The pillars of the scaffold may be rectangular, circular, oval, or another configuration to best fit the needs of that particular scaffold design. The scaffold inner rings may be parallel, concave to or convex to the outer edges of the anterior and posterior rings. Concave inner rings allow for anchoring mechanisms of the lenses to be positioned so that the anchoring mechanisms protrude between the scaffold and the interior of the capsular bag for effective anchoring of the lens. The scaffold preferably allows for rotational lens positioning to provide for accurate and stable toric lens correction for corneal astigmatism.

As discussed above, each ring of the scaffold of the present invention is preferably constructed to accept anchoring mechanisms of the add-in lens of the present invention, in a way to ensure reliable positioning in the capsule while making the insertion process relatively easy for the surgeon to manipulate. The add-in lens is accurately centered at a known position along the optical axis providing a stable refractive relationship among the optics, the cornea, and the retina allowing simplified and accurate measurements that deliver predictable, optimal visual acuity to the patient, depending upon the patient's specific refractive condition and the design and intended performance of each component of the multi-lens optical system.

It should be appreciated that the present invention teaches a heretofore unavailable flexibility offered to ophthalmic surgeons in lens selection and placement, even if their primary surgical results require explantation of the base lens. Any of the lenses, including the primary base lens, may be removed with relative ease and safety, and replaced with equally relative ease and safety, which is discussed, below.

It should be further appreciated that the described optical refinements afforded ophthalmologists by the present invention (1) occur in a pristine, clear, and healthy capsule free of adverse fibrosing and various opacifications (described herein) that result from the use of prior art 2-D IOLs and (2) benefit from optical design flexibilities created by an extended depth of focus in the primary lens and (3) permit implantation in capsules of varying sizes.

The aggregate of the aforementioned attributes is unique in the field of ophthalmology.

VI. Methods of Implantation

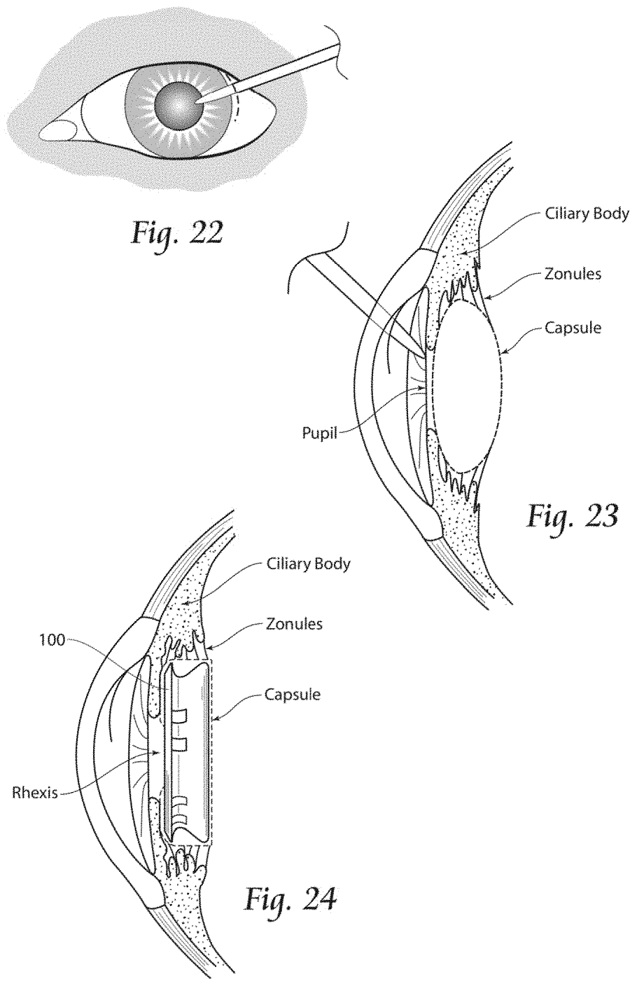

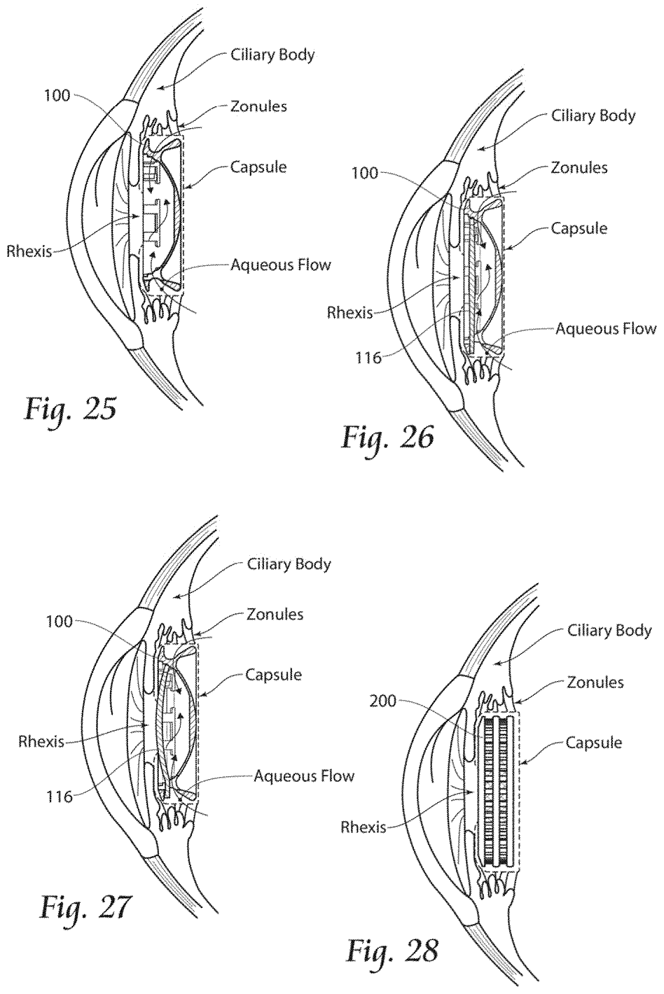

FIG. 22 shows an initial incision into the eye, so that the cloudy (cataractous) lens can be removed (FIG. 23). Once the cloudy lens has been removed, a scaffold is inserted and becomes centrally positioned within the lens capsule (FIG. 24). FIGS. 24-27 show the scaffold 100 after insertion, and FIG. 20 shows the scaffold 200 after insertion.

### Implantation of a Primary Lens Optic

The design of the scaffold of the present invention allows a primary optic to be affixed to the scaffold during manufacture prior to implantation within the eye, such that the scaffold and the optic may be inserted in a single surgical procedure. Alternatively, the scaffold may be inserted into the eye capsule and a primary lens optic inserted subsequently, either currently or during a future surgery, attaching the lens optic in a position within the capsule deemed desirable for the type of optic that may be inserted and affixed to the scaffold by the docking mechanisms. Absent the unpredictable post-surgical movement of ubiquitous 2-D IOLs, the scaffold preferably allows the effective lens position to be predicted with a high degree of precision and a primary lens more precisely selected (or created) based upon the desired post-surgical outcomes. This improved predictability for the present invention will enable a higher success rate for initial surgeries with a back-up option using an add-in lens to correct, or further optimize, initial surgical results as, once implanted, the position of the scaffold will be precisely known. There is no requirement that the primary optic be inserted within the same surgical procedure as the implantation of the scaffold, though the most likely option is implantation of a scaffold of the present invention inclusive of a primary lens. Because of the performance features of the scaffold, the scaffold provides the surgeon with considerable flexibility in deciding the best sequence of procedures and optical options for each patient.

### Implantation of Secondary or Tertiary Optic(s)

Following the initial cataract surgery, depending upon the position of the primary optic and the resultant optical assessments, the scaffold design allows the surgeon to be able to position one or two selected add-in lens optics anterior to the primary optic. That is, once the scaffold 100 is in place, the lens, e.g. lens 116, is positioned and anchored in place within the scaffold. As examples, FIG. 26 shows a planar lens, and FIG. 27 shows a convex lens, following a second implantation surgery.

The scaffold design provides for accurate placement of these lenses by properly affixing the add-in lens to the scaffold, as discussed above. The scaffold design allows the relationship between the lens optics of the present invention, i.e. the primary optic and the add-in lens 116, to be known, and, in turn, for the relationship between these lenses and the cornea and retina to be known prior to final, much simplified and reliable, computations before selection and implantation of the add-in lens, thus yielding optimal vision outcomes. The anchoring mechanism for the lens optics preferably preserves the physical and optical relationship between the lens optics such that they do not change over time relative to one another and to the cornea and retina.

The scaffold of the present invention allows for an add-in lens to enter a completely clear capsule essentially free of fibrosis and filled with clear aqueous fluid. These optimal conditions within the scaffold preferably provide that, should the surgeon and the patient determine that a different visual outcome is desirable, the secondary or tertiary add-in lens optics may be explanted and replaced with a minimal degree of complexity. The effective distance between any pair or combination of lenses can be managed by the placement of lenses at levels A, B or C (See FIG. 10A). The present invention as a 3-D IOL scaffold preferably allows for flexibility if primary surgery results require explantation of the primary lens. Any add-in lenses can preferably be removed with relative ease and replaced with equally relative ease.

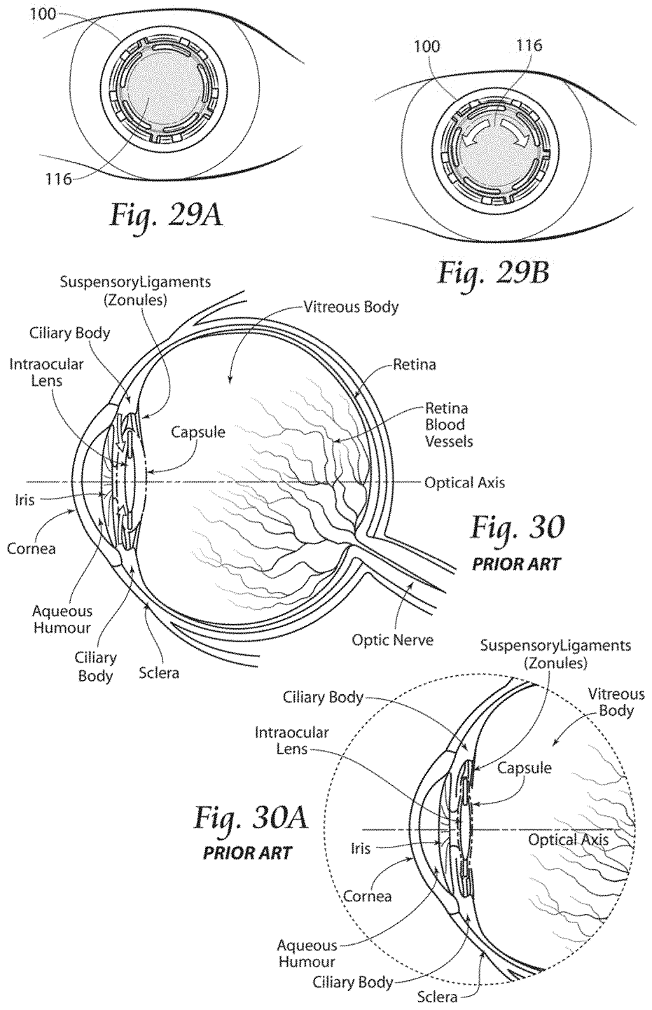

Similarly, the lens 116 can be adjusted, as discussed above, and shown in FIGS. 29A and 29B. The lens can be rotated either right or left and be repositioned and secured in place. Such positioning can be used to address an astigmatism once the scaffold has securely fibrosed to the capsule and further movement is unlikely.

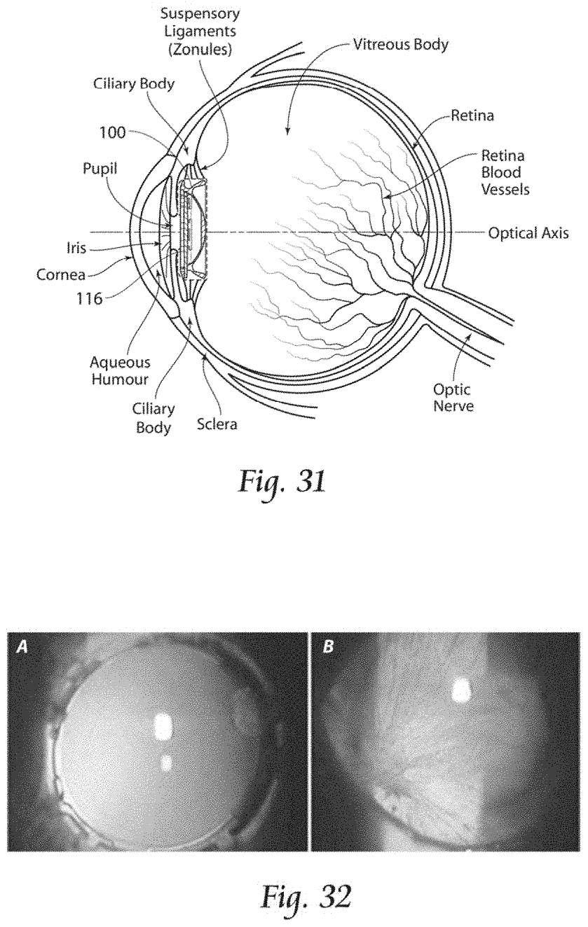

In comparison, a prior art implant (FIGS. 30 and 30A) is compared with the present invention (FIG. 31). As mentioned previously, the 3-D structure of the present invention, shown in FIG. 31, prevents the collapse of the capsule inwardly (see FIG. 30A vs FIG. 31) as the eye heals, providing a clearer pathway for light entering the eye, and avoids the lens dislocations (lateral, rotational and/or tilt) that accompany the fibrosing of prior art 2-D IOLs.

It is also apparent that the present invention can be used in other optical strategies. For example, one option to improve vision is through monovision, when the vision in the dominant eye is corrected for distance, and the other eye is intentionally left somewhat nearsighted. The resulting overlap of focal ranges provides an economic solution for patients who wish to be without eyeglasses but choose not to select premium IOLs. Best outcomes for this optical strategy necessitate a high degree of predictability of the refractive results for both eyes' IOLs. As described elsewhere in this application the present invention allows for more precise determination of lens placement that would enable the execution of a monovision option for patients compared to current surgical procedures using prior art IOL's.

As discussed herein, available cataract surgical procedures may struggle to optimize results for patients' low-order aberration needs such as hyperopia, myopia, presbyopia and astigmatism (far-sightedness, near-sightedness, age-related loss of focusing ability, irregular cornea shape). The present invention will not only address these situations in a superior manner, the precise optical solutions offered could then, and only then, be extended to effectively address higher order aberrations that can seriously impact vision such as spherical aberration that can reduce retinal image contrast and vision quality in low-light conditions.

VII. Benefits

### Allow Circulation of the Aqueous Humor

The structure of the scaffold has sufficient vertical and horizontal fenestrations in the two rings (anterior and posterior) and in the interconnecting pillars so as to allow the aqueous humor of the eye to circulate freely throughout the capsule. Before cataract surgery, and following surgery using 2-D IOL's, the aqueous humor circulates only in the anterior chamber of the eye. In the phakic eye (with the natural lens still in place in the capsule) the aqueous flows from the ciliary body and serves to hydrate, nourish and clean the anterior chamber (between iris and cornea), also delivering antibodies as and when necessary to counteract any infection (See FIGS. 1-3). Recent clinical studies have focused on the potential benefits of allowing the circulation of aqueous throughout the capsule of the aphakic (natural lens removed) eye. Preferably, the scaffold of the present invention, after implantation, maintains circulation of the aqueous humor, thereby maintaining ocular health of the entire anterior segment (anterior and posterior chambers), including preserving the natural suppleness of the capsule, and protecting the relationship between the capsule, the zonules, and the ciliary body. The scaffold specifically, and uniquely, affords aqueous circulation to the entire circumferential equator (fornix) of the capsule that is interconnected to the zonules and ciliary body (see Aqueous Flow Volume C in FIG. 10).

### Prevent or Minimize Capsular Fibrosis

Capsular fibrosis is associated with visual impairment in the aphakic eye. Fibrosis is a natural phenomenon of any trauma, effectively the development of scar tissue to help in the healing process. Cataract surgery, in the removal of the natural lens, requires first cutting a hole (rhexis) in the anterior lens capsule, then removal of the natural lens, both actions cause a certain amount of trauma in the eye. Capsular fibrosis is manifested by the creation of adhesions within the capsule. Implantation of 2-D IOLs (virtually all cataract surgeries) cause adhesion (1) of the anterior capsule to the posterior capsule where the two capsules are allowed to come into contact with each other, and (2) of the anterior and posterior capsules to the implanted lens optic and haptics—that is, adhesion of the capsule to any surface with which it comes into contact. Fibrosis of the eye capsule is associated with increasing the risk of zonular dehiscence, vitreous detachment, retinal detachment, lens decentration or tilt, any of which could require remediating surgical procedures such as lens removal that the presence of fibrosis necessarily complicates.

The scaffold of the present invention (3-D IOL) preferably prevents contact of the capsule with any portion of the device except the uppermost portion of the anterior ring and the outermost portion of the posterior ring (See FIGS. 10, 25-28). This means that the remainder of the capsule, being cleansed by the aqueous humor, and benefiting from 360 degree cell migration mitigation at the juncture of the capsule and the anterior and posterior rings, remains free of fibrosis and other epithelial-related cells that cause ACO, PCO or ILO (lens opacifications). Another benefit of the scaffold is that, with fibrosis occurring at the contact locales of the capsule and the scaffold rings, the scaffold over time becomes very stable in the eye, which means that refractive (along optical axis) and rotational stability (around optical axis) can be assured for (1) accurate re-assessment of any additionally required optics and (2) for the subsequent predictable placement of those optics within the capsule including toric lenses that are very sensitive to excessive rotation from targeted position.

### Minimize Posterior and Anterior Capsule, and Interlenticular, Opacification

Posterior capsule opacification (PCO) is caused by the migration of lens epithelial cells that are left on the anterior capsule to the equator (fornix) of the capsule, where they convert into blasts of lens cortical material. These blasts can then migrate along and to the posterior of the capsule between and behind a prior art 2-D IOL into the optical zone, effectively clouding the optical region and degrading visual acuity (see FIGS. 30, 32B, 33C, 33D). The surgical correction for PCO is to perforate the central optical zone of the posterior capsule using an Nd-YAG laser. Removal of the central optical zone of the posterior capsule gives rise to other potential health complications such as vitreous prolapse into the capsule, lens subluxation into the vitreous, and posterior capsule tears among others. Also, and commonly, patients affected by PCO, for a variety of reasons, do not obtain Nd-YAG laser surgery. Their vision remains compromised for the rest of their lives. Cataract surgery cannot be completely, and broadly, successful until and unless PCO is controlled.

The present invention's scaffold anterior ring 102 and posterior 104 ring (FIGS. 7-10, 21A-C) structure with its unique 360-degree capsule barrier at anterior and posterior ring edges prevents lens epithelial cell migration and blocks cortical fibers from encroaching into the posterior optical zone. Further, the base optic (if any) in this scaffold has minimal intersection with the posterior capsule—eliminating the cortical fiber capture zone that plagues 2-D IOLs. Any detached epithelial cells or cortical fibers cannot reattach and are borne away by the aqueous. (See FIGS. 10, 25-27.) The scaffold provides ample circulation of the aqueous humor, and the structure of an anterior ring and a posterior ring manages PCO and secures the scaffold in place.

Anterior capsule opacification (ACO) is caused by the capture of lens epithelial cells in the contact zone between the anterior capsule and the lens optic of prior art 2-D IOLs, which is manifested by the formation of Elschnig's pearls. ACO is generally credited with negative dysphotopsia, an ocular condition that results in the creation of blank or grey zones in the visual field, degrading visual acuity. The scaffold of the present invention preserves separation between the anterior capsule and any of the lens optics that may be placed within the scaffold, thereby preventing ACO and resultant lens cloudiness and negative dysphtopsias.

Interlenticular opacification (ILO) occurs, in a process similar to PCO development, between lenses that are implanted back-to-back with no, or minimal, separation. The present invention, scaffold-with-primary-lens plus add-in lens, is not prone to ILO as lenses are not in proximity to one another and incorporate fenestrations that permit aqueous circulation between the lenses.

### Structure

The aforementioned aqueous circulation, and fibrosing and opacification control, provide the ideal environment for successful cataract surgery. This results from the structure of the present invention (scaffold and add-in lens(es)) that further provides the framework for the implantation of additional lenses. As discussed previously, the structure of the scaffold of the present invention will flex to accommodate capsules of various sizes and fibrose into a fixed and ideal position that allows measurements of lens position along the optical axis among the primary lens, the cornea and the retina.

### Optical Design

A pristine environment, predictable lens positioning, and mounting framework are necessary conditions for the successful execution of an IOL design strategy. These are simultaneously provided by the present invention. As described herein, the present invention, following the initial implantation of the scaffold and its fibrosing in place within the capsule, provides for implantation of an add-in secondary lens along the optical axis at levels A, B or C and for rotation about that axis within the volume defined by the scaffold. A secondary lens can then be selected and implanted by a cataract surgeon to optimize visual results for the patient. In theory, it would be possible to write prescriptions for even more ideal optical solutions.

### Simplified, Precise Calculations

Present ophthalmological practice utilizes expensive diagnostic equipment and sophisticated formulae to calculate the power of the 2-D IOL that should be implanted for each eye of each patient. This process is confounded by physiological differences among patients, the eventual unpredictable movement of the 2-D lens as it fibroses in place, and the spectrum of capabilities and care levels among cataract surgeons. The same process would be followed for the initial implantation of the scaffold of the present invention (with a primary lens). However, the scaffold of the present invention naturally settles into a more square and centered position within the capsule due to its 3-D design. Unlike a prior art 2-D IOL, the primary lens of the scaffold of the present invention will ultimately rest in an advantageous and measurable position. Also, and equally important, the present invention will permit the implantation of a secondary lens that has been designed from known positions among the primary lens, the cornea and the retina using far more simplified formulae. Cumbersome, risky surgical techniques may be used to remedy relatively serious problems with prior art 2-D IOLs that have fibrosed in place. Other patients must tolerate imperfect results. The present invention will enable vision refinements to be executed for a much broader set of patients using simple surgical techniques.

### New Applications

Prior art 2-D IOLs, for many patients for reasons described herein, are not ideal for correction of lower-order aberrations (near-sightedness, far-sightedness or astigmatism). This precludes contemplation of other vision correction options or issues.

Monovision, different visual correction in each eye as discussed earlier herein, is not practical if relative lens powers cannot be closely managed. The present invention makes this practical.

Higher-Order aberrations can be addressed practically only if lower-order aberrations are resolved. The present invention creates a platform that will enable spherical aberration to be addressed, and it impact on vision quality in low-light conditions.

### Unique Combination

The present invention teaches a unique combination of products with an integral set of critically important ophthalmological attributes and methods of execution assembled in a manner heretofore unavailable to ophthalmologists. These products, attributes and methods are carefully delineated within this application. Some similar products and methods are described in the industry but not in this powerful combination of the present invention.

VIII. Results

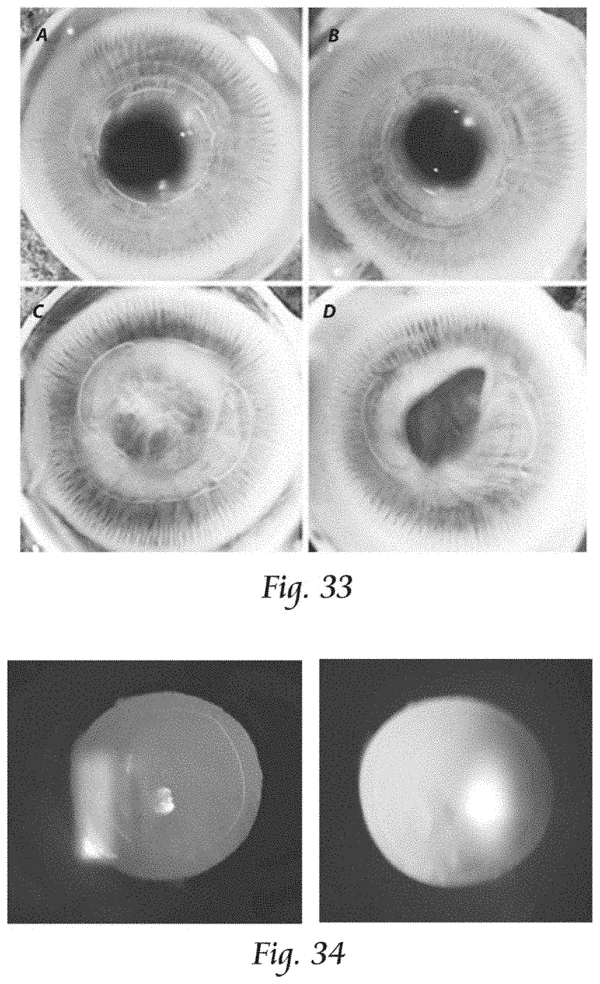

Testing was undertaken within rabbits to determine the capsular clarity of the present invention (without add-in lens) to the prior art. FIGS. 32 and 33 are representative of results depicting relative capsule clarity from numerous six-month rabbit studies conducted over many years (2010-2022). In FIG. 32, the present invention (left photo) compared very favorably to the prior art (right side). These slit-lamp photos (anterior view of living rabbits) of the retina were taken six months following implantation. The six month period for a rabbit is considered in the industry as a representative model of many years following implantation in a human.

In FIG. 33, post-mortem, posterior photos of the capsule of dissected eyes A and B (present invention) compare very favorably to C and D (prior art).

In FIG. 34, the slit lamp photos (anterior view) are different exposures of the same eye of a human clinical trial patient 36 months following surgery using the present invention and both demonstrate comparable and important results to photo A of FIG. 32 regarding long-term capsule clarity in human eyes.

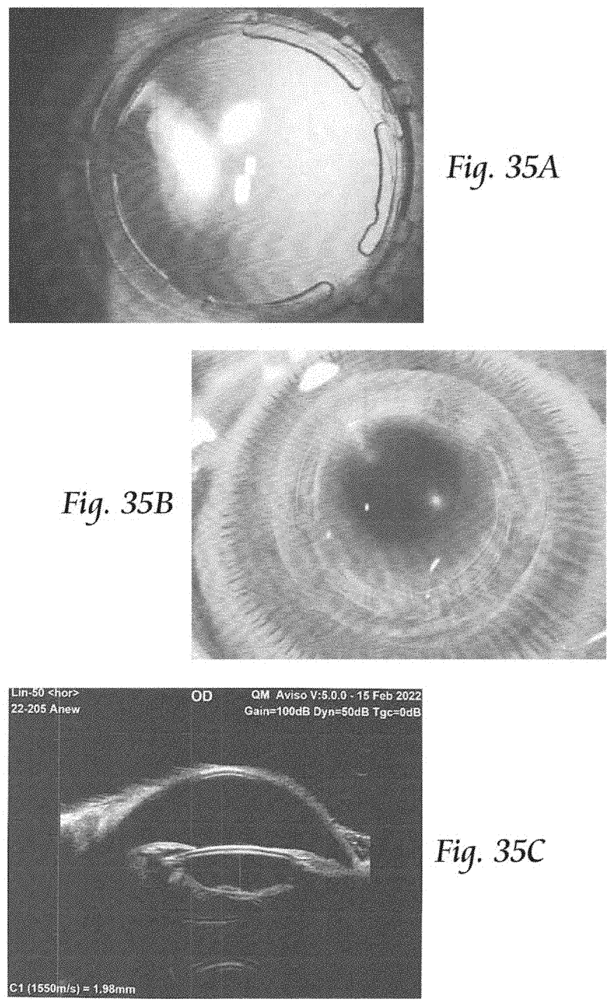

FIGS. 35A-C show the photographic results of a five-week week rabbit study of the present invention including an add-in lens (as shown in FIGS. 16 and 17A). The various views include the add-in lens in an anterior view (FIG. 35A), the scaffold in a post-mortem dissection posterior view (FIG. 35B), and in a lateral view MRI that depicts the primary lens of the scaffold at the bottom and the add-in secondary lens at the top (FIG. 35C).

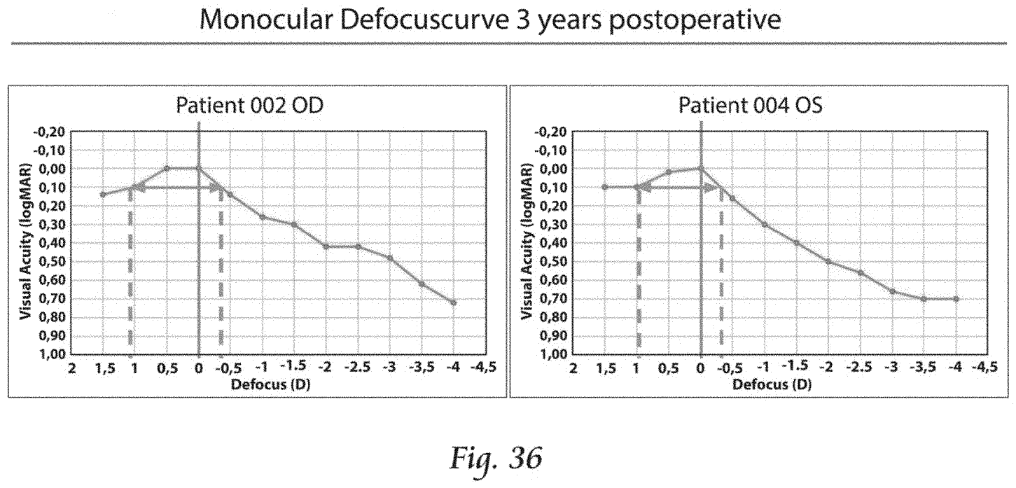

Tn FIG. 36 the three-year results of a human clinical study of two patients are depicted. The industry-accepted methodology (defocus curves) was utilized to measure the focusing range of the primary lens of the present invention and is reflected in the hashed lines. It is believed this extended depth of focus (EDOF) results from the potential movement of the primary lens in response to ciliary eye muscle prompts (accommodation) and/or from pseudo-accommodation due to the design characteristics of the primary lens and/or its position near the posterior capsule. This attribute provides further important optical design flexibilities for add-in lens of the present invention. The present invention may be modified to provide even more extended depth of focus. Further benefit may accrue to the present invention as preliminary indications of accommodation by the primary lens are borne out.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. An intrascapular scaffold for insertion into an ocular capsular bag to replace a human lens during cataract surgery and capable of receiving a secondary lens, said scaffold comprising:
an anterior ring;
a posterior ring;
at least two pillars coupling said anterior ring and said posterior ring;
a primary intraocular lens secured to one of said anterior and posterior rings;
horizontal fenestrations located within an inner edge of said posterior ring to provide for circulation of the aqueous humor through two non-equatorial regions of the ocular capsular bag; and
a plurality of vertical fenestrations between said pillars, said fenestrations intersecting said anterior and said posterior ring and said horizontal fenestrations, said vertical fenestrations adapted to receive said secondary lens.

2. The intrascapular scaffold of claim 1, wherein the rings and the pillars are of the same material.

3. The intrascapular scaffold of claim 1, wherein vertical fenestrations provide for additional circulation of aqueous humor to an entire circumferential equatorial region of the ocular capsular bag.

4. The intrascapular scaffold of claim 1 further comprising:
an interior ring positioned between said anterior ring and said posterior ring.

5. The intrascapular scaffold of claim 4, wherein the diameter of said interior ring provides a convex curvature to said pillars.

6. The scaffold of claim 5, wherein the diameter of said interior ring is equal to the diameter of said anterior and posterior rings.

7. An implant for an ocular capsular bag to replace a human lens of the eye capsule removed during cataract surgery, said implant comprising:
a scaffold, said scaffold comprising:
an anterior ring;
a posterior ring positioned away from said anterior ring;
at least one interior ring positioned between said posterior ring and said anterior ring;
a plurality of pillars coupling said anterior ring and said posterior ring and said interior ring;
a primary lens permanently secured to one of said posterior ring or said anterior ring;
a plurality of horizontal fenestrations located within the inner edge of said posterior ring to provide aqueous humor circulation within the capsule through two non-equatorial regions of the ocular capsular bag;
a plurality of vertical fenestrations between said pillars, said fenestrations intersecting said anterior, said posterior ring, said interior ring and said horizontal fenestrations, said vertical fenestrations enabling aqueous humor circulation;
a plurality of tabs positioned at the inner edge of said interior ring;
a secondary lens having one or more anchoring mechanisms extending outwardly from said secondary lens;
said secondary lens having a plurality of fenestrations located near said outer edge of said secondary lens; and
said secondary lens being removably attached to one of said anterior ring, said posterior ring, or said at least one interior ring with said anchoring mechanisms being inserted into one of said vertical fenestrations or onto said tabs, wherein said attachment of said secondary lens defines open volumes within said scaffold on either side of said secondary lens, thereby maintaining said circulation of the aqueous humor on either side of said secondary lens through said secondary lens fenestrations.

8. The implant according to claim 7, where said posterior ring or said anterior ring flexes in response to movements of the ciliary muscle of the eye after implantation via zonules, thereby changing the curvature and power of said primary lens and to extend the depth of focus of said primary lens.

9. The implant according to claim 7, wherein said posterior ring and said anterior ring are shaped to allow said eye capsule to shrink around said posterior ring and said anterior ring, thereby precluding migrations of epithelial cells within said eye capsule.

10. The implant according to claim 7 wherein said flexing of said anterior or said posterior ring is in response to said eye capsule shrinking around scaffold, thereby allowing said implant to have a secure fit for any variable size of said eye capsule.

11. The implant according to claim 7, wherein said interior ring comprises a shelf within said scaffold, said secondary lens being attached to said shelf.

12. The implant according to claim 7, wherein said scaffold comprises at least two interior rings, each of said interior rings comprising a shelf, said secondary lens being attached to one of said shelves.

13. The implant according to claim 7, wherein said vertical fenestrations are asymmetrically arranged.

14. The implant according to claim 13, wherein there are at least five horizontal fenestrations.

15. The implant according to claim 14, wherein there are at least 10 vertical fenestrations.

16. The implant according to claim 15 wherein said secondary lens is associated with a prescribed optical meridian and inserted in an appropriate vertical fenestration for addressing astigmatism correction.

17. The implant according to claim 16, wherein said secondary lens has an optical meridian, said secondary lens optical meridian being set off axis of one of said prescribe optical meridians of said vertical fenestration when attached to said scaffold.

18. The implant according to claim 7, wherein said secondary lens is concave.

19. The implant according to claim 7, wherein said secondary lens is convex.

20. The implant according to claim 7, wherein said secondary lens is flat.

21. The implant according to claim 7, wherein the diameter of said interior ring provides a convex curvature to said pillars.

22. The implant according to claim 7, wherein the diameter of said interior ring provides a concave curvature to said pillars.

23. The implant according to claim 7, wherein the diameter of said interior ring is rectilinear to said pillars.

24. A method for replacing a lens of the eye capsule, the method comprising the steps of:
removing said lens from the eye capsule;
providing an insert according to claim 7; and
anchoring said insert within said eye capsule.

25. The method according to claim 24 further comprising the steps of:
assessing the visual acuity of the eye; and
attaching said secondary lens to said scaffold at a first position based on the assessment of the eye.

26. The method according to claim 25 further comprising the steps of
reassessing the visual acuity of the eye;
removing said secondary lens from said scaffold if needed based on reassessment;
rotating said secondary lens to a second position;
reattaching said lens to said scaffold, wherein the rotating and reattaching of said lens provides toric correction for corneal astigmatism.

27. The method according to claim 25, wherein said secondary lens is selected based upon precalculated optical characteristics.

28. The method according to claim 25, wherein said steps of removing, rotating, and reattaching said secondary lens are performed directly after anchoring said insert within said eye capsule.

29. The method according to claim 25, wherein said steps of removing, rotating, and reattaching said secondary lens are performed at a different time than anchoring said insert within said eye capsule.

30. The method according to claim 28 further comprising the steps of:
explanting said secondary lens from said implant; and
attaching another secondary lens to said implant.

31. The method of claim 25 wherein said first position is located on said anterior ring and said second position is located on said at least one interior ring.

32. The method of claim 25 wherein said first position is located on said at least one interior ring and said second position is located on said anterior ring.

33. The method of claim 24, wherein said method is directed towards a monovision procedure.

34. The method of claim 24, wherein said method is directed towards correction of a spherical aberration and the correction of low-light difficulties.

35. The method of claim 24, wherein said implant provides pseudoaccomodation.
